(19) United States
(12) Patent Application Publication
(10) Pub. No.: US 2026/0077146 A1
(43) Pub. Date: Mar. 19, 2026

(54) TRACHEAL INTRODUCER SHEATH AND SIMPLIFIED TRACHEAL INTRODUCER SHEATH

(71) Applicant: Christopher Taicher, Ojai, CA (US)

(72) Inventor: Christopher Taicher, Ojai, CA (US)

(21) Appl. No.: 19/393,112

(22) Filed: Nov. 18, 2025

Related U.S. Application Data
(63) Continuation of application No. 17/567,715, filed on Jan. 3, 2022, now Pat. No. 12,496,416.
(60) Provisional application No. 63/283,455, filed on Nov. 27, 2021.

(51) Int. Cl. A61M 16/04

(52) U.S. Cl. CPC A61M 16/0418; A61M 16/0488; A61M 2205/0216; A61M 2205/0266; A61M 2205/582

(57) ABSTRACT

According to the CDC, the same month (March, 2020) they declared COVD-19 a pandemic, nearly 25% of confirmed COVID-19 hospitalized patients required intubation, or ventilator use. More recently, in the US, during the summer of 2021, child intubations more than quadrupled, when the Delta variant predominated.

As Omicron and additional COVID-19 variants continue to arise globally, there will be an increasing number of sudden spikes in critical illness and respiratory failure. Preparedness with efficacious airway tools that optimize success rate of intubations is critical.

Without the proper tools, intubation procedures take excessive time, and can fail entirely, causing diminished central nervous system oxygenation and potentially permanent neurologic sequelae. Airway adjuncts such as the Tracheal Introducer Sheath (TIS) and Simplified Tracheal Introducer Sheath (STIS) hold immense potential to improve chances of successful intubations.

The TIS is a STIS with an added articulating mechanism, but otherwise is a similar device. For the purposes of further explanation, the TIS refers to both TIS and STIS unless otherwise specified.

TIS is an adjunct medical device for control and guidance of any form of tracheal introducer device through the glottic opening of a patient's airway. This facilitates endotracheal intubation by passage of the endotracheal tube.

An airway introducer is placed within and controlled by the TIS, a mechanically specialized sheath, open at both ends, and covering the distal end of the introducer (where control is most relevant). The TIS acts as a relatively stiff channel, or tunnel, for the introducer to be directed through for smooth travel along courses of varied angles. The proceduralist is able to apply curvature in real-time to the TIS-introducer assemblage based on patient anatomy and other clinical factors. Without the TIS as a channel, an introducer alone can be bent at its tip, but it is limited in its ability to move along that axis as it is being proximally controlled, outside of the patient.

The mechanistic basis for the sheath's guidance capability is based on composition, including, but not limited to, a hollowed shaft of polytetrafluoroethylene (PTFE), with a specific ID/OD (internal diameter/outer diameter) ratio, such that there is sufficient stiffness for the sheath to act as a guide within which the introducer can travel, whilst maintaining appropriate elasticity for malleable control.

The TIS confers additional introducer controllability by minimizing introducer wobble, as introducers can consist of relatively long shaft-like devices, often nearly 60 cm.

Introducer guidance by a TIS, but not the simplified version (STIS) includes a distal articulating tip mechanism, angled as needed while in the tracheal space of a patient, and controlled outside of the patient by moving the proximal sheath through a pull sheath mechanism. To recap, the Simplified Tracheal Introducer Sheath (STIS) lacks this articulating mechanism, but otherwise is a similar device.

Rotatory control in the case of both the TIS and STIS is achieved by rotating the introducer which sits outside the proximal end of the sheath.

A major additional benefit of utilizing a sheath with an introducer is selection of an introducer for placement into a sheath on a case-by-case basis. For example, a smaller, softer tipped introducer could be utilized with a TIS in cases where airway friability or injury is of particular concern; i.e. a mass with high vascularity and bleeding potential. In another example an introducer with a central canal for oxygenation can be utilized with a TIS when the decision is made to oxygenate through the introducer prior to placement of the endotracheal tube.

Finally, the TIS, with its integration of an articulating mechanism utilizing a pull sheath is without costly electrical components and thus serves as a cost-effective alternative to fiberoptic intubation with a bronchoscope (often utilized among top academic hospitals), which is often cost prohibitive for many rural and community hospitals to stock. As such, the TIS may improve access of lower socioeconomic classes to important technologies in the management of respiratory failure.

### CROSS-REFERENCE TO RELATED APPLICATIONS

This application is a continuation of U.S. application Ser. No. 17/567,715, filed Jan. 3, 2022, which claims the benefit of U.S. Provisional Application Ser. No. 63/283,455, filed Nov. 27, 2021. The disclosure of the prior applications is considered part of (and is incorporated by reference in) the disclosure of this application.

### STATEMENT REGARDING FEDERALLY SPONSORED RESEARCHED OR DEVELOPMENT

Not Applicable

### THE NAMES OF THE PARTIES TO A JOINT RESEARCH AGREEMENT

Not Applicable

### INCORPORATION-BY-REFERENCE OF MATERIAL SUBMITTED ON A COMPACT DISC OR AS A TEXT FILE VIA THE OFFICE ELECTRONIC FILING SYSTEM (EFS-WEB)

Not Applicable

### STATEMENT REGARDING PRIOR DISCLOSURES BY THE INVENTOR OR A JOINT INVENTOR

Not Applicable

### BACKGROUND OF THE INVENTION

Severe and critical cases of COVID-19 raise clinical concerns for progression to respiratory failure and a need for airway protection with endotracheal intubation. [What is endotracheal intubation? A procedure typically requiring utilization of a laryngoscope blade in one hand to help visualize the vocal cords (“cords”) and glottic opening for passage (with the opposite hand) of an endotracheal tube into the tracheal space.]

Since nearly 20% of past COVID-19 cases were considered severe or critical, according to a large 2020 study in the Journal of the American Medical Association, the U.S. healthcare system must be prepared for spikes in COVID-19 incidence and related increases in frequency of airway failures. The pediatric COVID-19 population characteristics underscore this point: Among 20,714 hospitalized children and adolescents diagnosed with COVID-19, 31% experienced severe COVID-19.

There is an additional problem that proceduralists (emergency medicine physician, anesthesiologist, ambulance personnel, etc.) face: the so-called “difficult airway”, or those that pose technical challenges.

Difficult airways represent up to nearly one-quarter of all intubations and according to the American Journal of Respiratory and Critical Care Medicine, are due mostly to a difficulty placing the endotracheal tube. This can be for many reasons including difficulty visualizing the cords due to edema (a well described complication with COVID-19-associated acute respiratory distress syndrome, or ARDS), foreign body, a malignant mass, or simply patient's baseline anatomy. The outcomes of COVID-19-associated ARDS patients are especially grim, with a mortality of 45%, and thus this population deserves special attention in the U.S. research and development spaces.

Regardless of the cause, complicated intubation procedures that take excessive time (with multiple “passes” or attempts at passing an endotracheal tube) are associated with diminished central nervous system oxygenation and potential neurologic disease.

As such, much effort has been put towards improving likelihood of first attempt (“first pass”) success during expectedly and unexpectedly difficult intubations. The most notable of airway adjuncts for this purpose is the Bougie Device, patented in 2007 (U.S. Pat. No. US20100307489Al).

Introducers, like the Bougie Device, tend to be helpful because of their relatively small size and (depending on device) kinked and malleable distal tip as compared to an endotracheal tube. Once an introducer is passed through the cords, sometimes blindly (again, due to severe pathology such as edema, or anatomy, or both), tracheal positioning of an introducer is confirmed by feeling tracheal rings as vibratory feedback in the hand operating the introducer. Subsequently, an endotracheal tube is passed over the introducer, and the introducer removed from the patient, for endotracheal tube securing and then attachment to a mechanical ventilator.

Given strong evidence on first-attempt intubation success being significantly higher when using an introducer, intubation proceduralists will likely be continuing to utilize introducers.

The most notable problem with existing introducers, including the Bougie Device, is its limited ability to maintain desired contour and, once they have been placed beyond the oral cavity and oropharynx, take a sharp angle around an often obstructing epiglottis to move through the cords. A kinked or angled introducer tip is extremely limited in taking steep angles because it is pushed by the proceduralist with a force vector driven mostly external to the patient. Sometimes the proceduralist is controlling the introducer within the patient's oropharynx, but this still is relatively external and angled away from the deeper glottic opening. Thus, curving the tip of an introducer that is deep in the patient's airway confers only so much ability to take a steep angle. However limited, an operator can also move the introducer along a curved plane to add some improved angular control.

With both the TIS and STIS, and relatively tiff sheath through which an introducer can move allows the introducer to move along the axis created by the sheath itself, which can be molded quickly to the desired angle, regardless of steepness, up to ninety degrees.

As mentioned, manually molding a kink of varied angles at the tip of an introducer prior to introduction into the patient is common practice to aid its navigation towards and through the cords, however this corrective action has limited utility, including an inability to manipulate the angle once placed into the patient.

The TIS solves this problem by taking on varied angles (articulating between 10 and 90 degrees), in real time, controlled at the proximal end of the TIS (outside of the patient), whilst the distal introducer is placed deeper in the airway.

While a patent pending articulating device (Total Control Introducer by Through The Cords LLC) includes an articulating mechanism, the device is itself an introducer. Uniquely, the TIS is a sheath through which a broad range of introducers can be controlled. As such, varied introducer size, material, shape and design (i.e. ones with a central lumen for oxygenation or blunt-closed-end tips) can be chosen on a clinical case-by-case basis for utilization with a TIS. For example, a patient with an especially friable airway can be intubated using a TIS and an especially soft tipped introducer to avoid potentially injuring the soft tissue and causing acute and chronic complications, such as bleeding and fistula creation, respectively. Any number of introducer types can be designed and manufactured for assembly with a TIS.

In addition to a TIS allowing for selection of varied introducer types on a clinical case-by-case basis, it eliminates waste of existing supplies of introducers, and supports continued, specialized and streamlined manufacturing.

Another issue with known introducers, Bougie, Frova and others, are their relative unwieldy form. Given they are composed most commonly of thin (commonly approaching 6 mm), lengthy (often between 50 and 70 centimeters), PTFE, they produce a somewhat flimsy structure. This is of particular concern during acute and emergency settings wherein patient and environmental factors require steadied instrumentation. Introducers of this length will wobble in free space above the patient, which transfers to the distal introducer (inside the patient). This can become a similarly haptic challenge at the proximal end when attempting to place and slide a endotracheal tube over the introducer.

TIS solves the unwieldiness of most introducers by sheathing them in material with relative stiffness (increased elasticity modulus through), including but not limited to PTFE with a specified ID/OD ratio (see below).

The invention will be more fully understood by reference to the following descriptions accompanying drawings.

### FIELD OF THE INVENTION FOR AIRWAY INTUBATION INVOLVING TRACHEAL INTRODUCERS

### BRIEF SUMMARY OF THE INVENTION

The present invention is an airway adjunct that allows for ease of passage of an introducer through the glottic opening. The placement of an introducer may be utilized to assist with the ultimate goal of endotracheal intubation, or for whatever the primary goal of the proceduralist, including, but not limited to placing an introducer primarily for emergent oxygenation through the introducer with central canula.

An unprecedented benefit of this invention is the ability to select an introducer on a clinical case-by-case basis (of varied size, style, shape and design) for sheathing into the TIS. An introducer sheathed in an TIS offers the proceduralist substantial improvement in haptic control, including improved maneuverability, remote distal articulating control, an improved force vector by sliding an introducer over a relatively stiff distal channel, and thus overall ease of use and safety profile. This device supports manufacture of introducers by manufacturers who maintain particular knowledge and streamlined operations of introducers of varied material, specialty and function.

### BRIEF DESCRIPTION OF THE SEVERAL VIEWS OF THE DRAWING(S)

FIG. 1 is a schematic illustration of a human airway with a tracheal introducer sheath device with introducer placed within (also referred to as ‘sheath-over-introducer assemblage’).

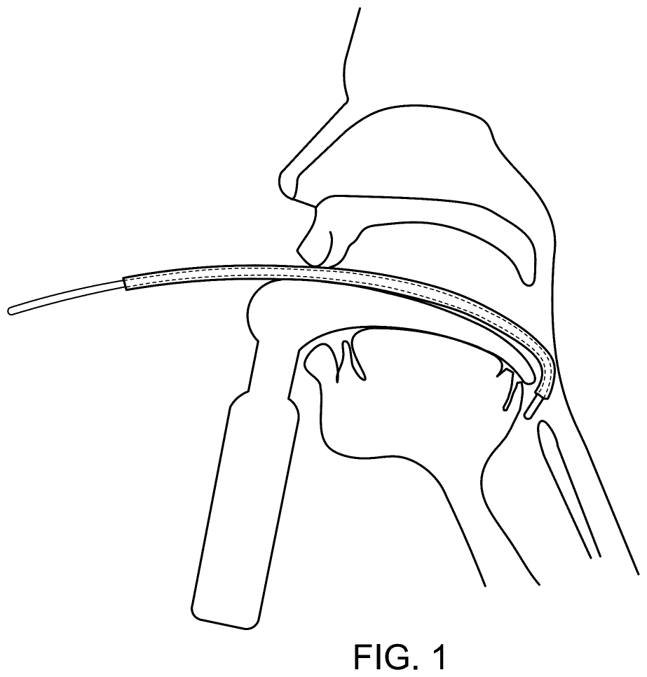

FIG. 2 shows the tracheal introducer sheath device.

FIG. 3 illustrates an introducer device placed within the sheath of the tracheal introducer sheath.

FIG. 4 illustrates an axial view of a TIS (but not a BTIS) from proximal and distal ends (above and below, respectively), showing vertebrated succession rigid annuli.

FIG. 5 is a schematic illustration of a human airway with laryngoscope assisted insertion of a sheath-over-introducer assemblage into the trachea.

FIG. 6 is a schematic illustration of a human airway with laryngoscope assisted advancement of an introducer through the tracheal introducer sheath device until making contact with tracheal rings.

FIG. 7 is an illustration of a human airway with correct placement of an introducer in the trachea after a tracheal introducer sheath device has been removed.

FIG. 8 is a schematic illustration of a human ainvay with correct placement of an endotracheal tube over an introducer and into the trachea.

FIG. 9 illustrates the resulting position of the endotracheal tube after removal of an introducer.

### DETAILED DESCRIPTION OF THE INVENTION

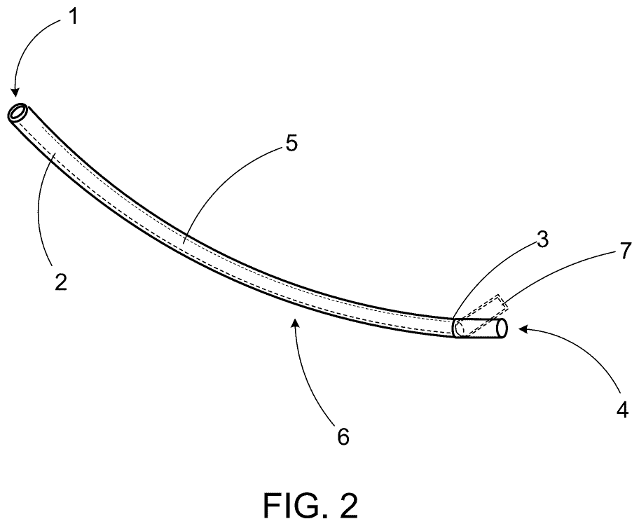

FIG. 2 of the drawings illustrates a preferred embodiment of the invention which has been referred to hereinabove as a tracheal introducer sheath (TIS), since the instrument 6 is applicable to assisting passage of a tracheal introducer 10 into the ainvay, through the oropharynx 9 and past the glottis, for endotracheal intubation. It is to be understood, however, that instrument 6 is not limited to the aforementioned uses. It may, for example, be used for sheathing an introducer 10 of many types such as but not limited to one with a central lumen, for emergent ainvay oxygenation.

Instrument 6 comprises a main portion of the body 5, connecting an open-ended sheath (proximal opening 1 and distal opening 4) by a hollowed tube of PTFE, twelve to fifty centimeters long, with varied ID/OD ratios depending on the chosen length.

As a point of clarification, the length of each individual instrument 6 will generally not be modifiable. A notable functional exception would be if an introducer 10 manufacturer produced an introducer 10 with modifiable length. In this case, if the introducer 10 is placed through the instrument 6, length would be modifiable of the sheath-over introducer assemblage.

For an adult instrument 6 of length between twelve and thirty centimeters long, the instrument 6 is to have an internal diameter (ID) ranging between 4 mm and 7 mm with outer diameter (OD) ranging between 6 mm and 9.5 mm. Tube thickness for an adult instrument 6 is to range from 0.5 to 3 mm. One embodiment for an adult size instrument 6 is 20.5 centimeters long with an ID of 6 mm and an OD of 8.4 mm, with a wall thickness of 1.2 mm.

For the same instrument 6 sized for children, the instrument 6 length is to range from between seven to fifteen centimeters long with IDs ranging between 2.5 mm and 5 mm. Outer diameters will range between 4.5 mm and 7 mm. Tube thickness for a pediatric version of instrument 6 is to range from 0.2 to 2 mm.

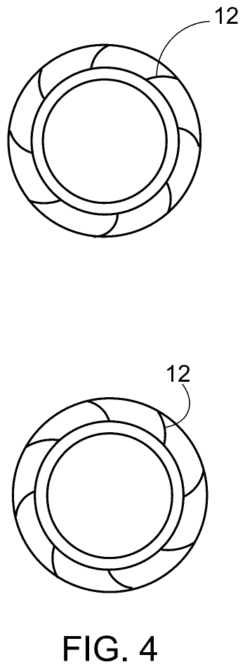

For the TIS but not the STIS, operator controlled articulation of the articulating component 3 occurs by applying a pull force to the proximal portion 2 of the instrument 6, which transmits along an internally installed vertebrate succession of rigid annuli 12 from the proximal portion 2 of the instrument 6 through the body 5 and into the articulating component 3 of the tip and distal opening 4, allowing for proximal operating wires (illustrated in FIG. 4) to effect a smoothly operating graduated bending over a substantial length of the instrument's 6 distal portion, without effect upon intermediate portion of the body 5 and proximal portions 2 of the sheath 6. The vertebrate succession of annuli 12 seen in FIG. 4 is installed internally to the smooth internal surface of the sheath, maintaining smooth passage of an introducer. For those interested in greater detail in the construction of a pull-sheath that maintains a uniformly diametrically dimensioned internal hollow cavity throughout a tube within which fiber bundles are extended, allowing simultaneous proximal-to-distal control of an articulating mechanism, see U.S. Pat. No. 3,913,568.

The articulating component 3 allows angulation 7 between five and ninety-five degrees.

The size of the internal diameter of the instrument is constant throughout each individual instrument 6 but can vary between different instruments 6, allowing for passage of different sized introducers 10 when desired by the proceduralist.

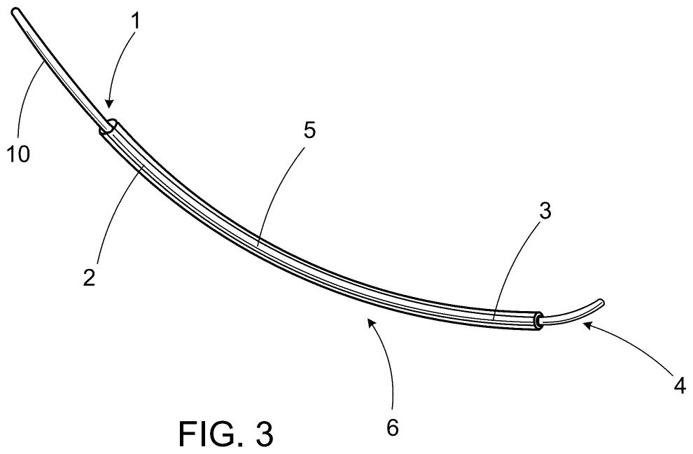

FIG. 3 Illustrates a sheath-over-introducer assemblage, emphasizing both the adjunct nature of the instrument 6, to be utilized with an introducer 10, as well as the 360-degree free rotational movement of an introducer within an instrument 6.

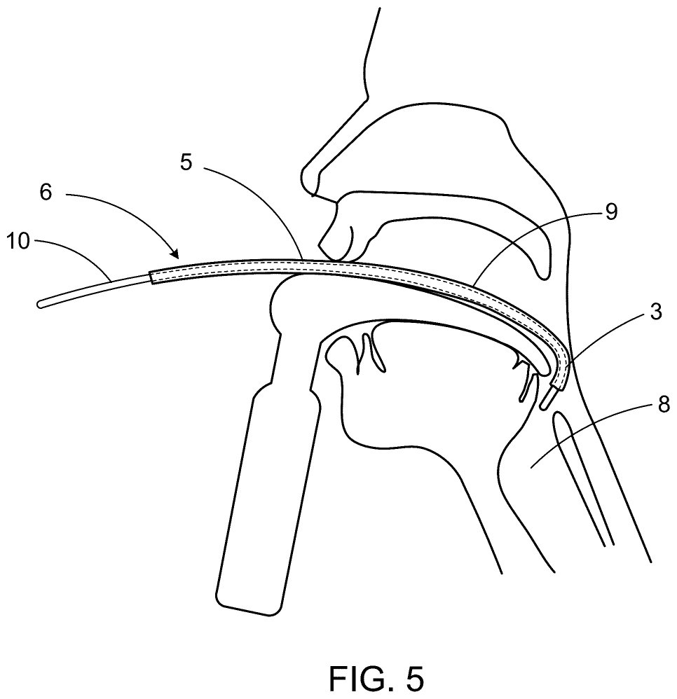

The first steps of operating a tracheal sheath introducer 6 are illustrated in FIG. 5, during which a proceduralist uses either direct laryngoscopy or video-assisted laryngoscopy to insert a tracheal introducer sheath device 6 with introducer into the airway.

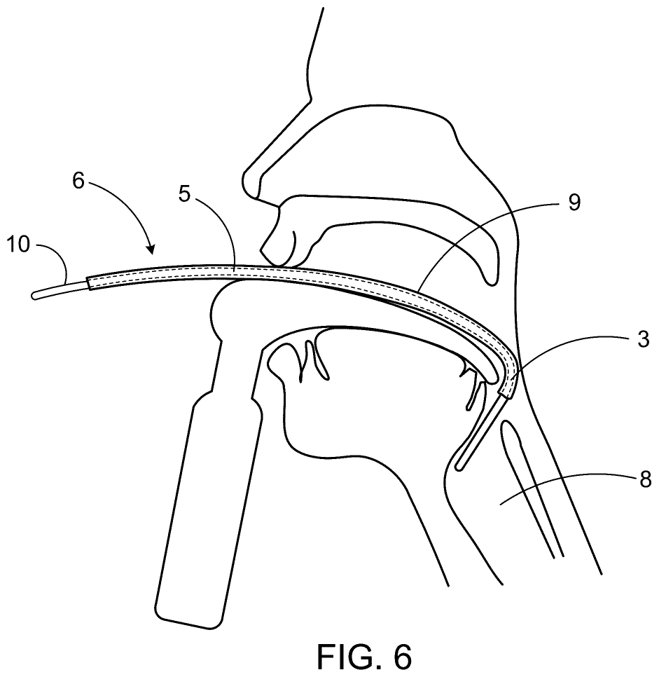

Once in appropriate proximity to the glottic opening, the tracheal introducer sheath device 6 is held in place, acting as a supporting vehicle through which the introducer is passed and directed towards the tracheal rings 8 (FIG. 6) until a vibratory sensation is felt that reflects tracheal ring 8 resistant forces have been met. Given the need to hold the laryngoscope and the sheath instrument 6, a third person trained in this procedure will advance the introducer 10 at the instruction of the main operator or proceduralist (holding the laryngoscope). Conversely, the proceduralist may ask an appropriately trained assistant to hold the sheath instrument 6 whilst the proceduralist advance the introducer 10.

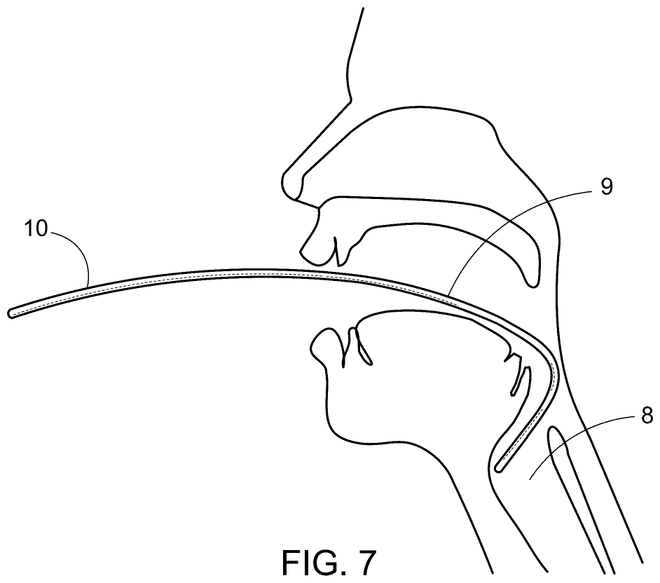

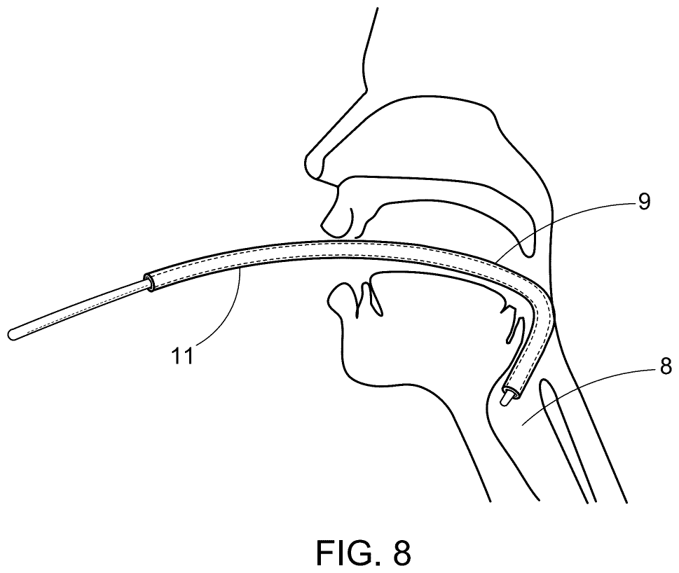

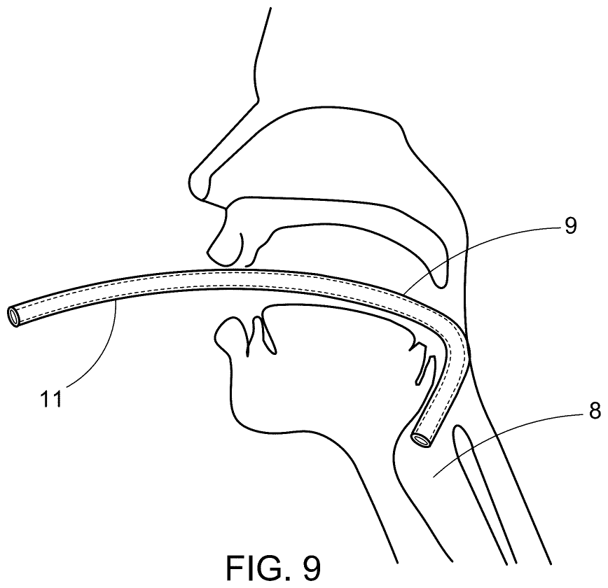

At this point, the introducer 10 is held in place while the instrument 6 can be removed (FIG. 7) and an endotracheal tube 11 subsequently slid over the introducer 10 as in FIG. 8. The introducer is removed and endotracheal left in place (FIG. 9) for subsequent securing and mechanical ventilation.

1-9. (canceled)

10. An assembly for assisting placement of an endotracheal tube into the airway of a person, comprising:
a tracheal introducer sheath comprising a sheath channel that extends from a proximal end of the sheath to a distal end of the sheath through which a tracheal introducer removably extends, wherein an inner surface material of the tracheal introducer sheath that defines the sheath channel is relatively stiff in comparison with a stiffness of the tracheal introducer and wherein the tracheal introducer sheath is manually moldable to articulate different angles.

11. The assembly of claim 10, wherein the tracheal introducer sheath has a wall thickness of 0.2 mm to 2.0 mm.

12. The assembly of claim 10, wherein the tracheal introducer sheath's length is between twelve and fifty centimeters to provide coverage of the length of the tracheal introducer.

13. The assembly of claim 12, wherein the length of the tracheal introducer sheath is 20.5cm.

14. The assembly of claim 10, wherein the tracheal introducer sheath comprises a vertebrate succession of rigid annuli from the proximal end of the sheath to the distal end of the tracheal introducer sheath.

15. The assembly of claim 10, wherein the tracheal introducer sheath includes operating wires along its length which provide means for gradually bending the tracheal introducer sheath through application of a pull force to a portion of the tracheal introducer sheath adjacent to the proximal end of the tracheal introducer sheath.

16. The assembly of claim 10, wherein a ratio of an outer diameter of the sheath and an inner diameter of the sheath is based on a chosen length of the sheath.

17. The assembly of claim 10, wherein a ratio of an outer diameter of the sheath and an inner diameter of the sheath is uniform along a chosen length of the sheath.

18. A method for guiding insertion of a tracheal introducer for subsequent placement of an endotracheal tube into the airway of a person, the method comprising:
providing a tracheal introducer sheath that defines a sheath channel along the length of the sheath through which the tracheal introducer removably extends, wherein an inner surface material of the tracheal introducer sheath that defines the sheath channel is relatively stiff in comparison with a stiffness of the tracheal introducer and wherein the tracheal introducer sheath is manually moldable to articulate different angles;
bending the tracheal introducer sheath into a shape for desired placement of a distal end of the sheath within the airway;
inserting the tracheal introducer sheath into the airway;
guiding the tracheal introducer into a glottic opening of the person's airway via the tracheal introducer sheath;
removing the tracheal introducer sheath from the tracheal introducer when a distal end of the tracheal introducer is determined to be properly placed within the airway;
positioning an endotracheal tube over the tracheal introducer; and
removing the tracheal introducer from the airway after positioning the endotracheal tube.

19. The method of claim 18, wherein the tracheal introducer sheath has a wall thickness of 0.2 mm to 2.0 mm.

20. The method of claim 18, wherein the tracheal introducer sheath's length is between twelve and fifty centimeters to provide coverage of the length of the tracheal introducer.

21. The method of claim 20, wherein the tracheal introducer sheath's length is 20.5cm.

22. The method of claim 18, wherein the tracheal introducer sheath comprises a vertebrate succession of rigid annuli from the proximal end of the tracheal introducer sheath to the distal end of the tracheal introducer sheath.

23. The method of claim 22, including the step of applying a pull force to a portion of the tracheal introducer sheath adjacent to the proximal end of the tracheal introducer sheath, to affect a gradual bending along the length of the tracheal introducer sheath.

24. The method of claim 18, wherein a ratio of an outer diameter of the sheath and an inner diameter of the sheath is based on a chosen length of the sheath.

25. The method of claim 18, wherein a ratio of an outer diameter of the sheath and an inner diameter of the sheath is uniform along a chosen length of the sheath.
